(19) United States
(12) Patent Application Publication
(10) Pub. No.: US 2002/0193856 A1
(43) Pub. Date: Dec. 19, 2002

(54) Water bag with new structure for cold / hot hydrotherapy

(76) Inventor: Nan Chih Lu, Pingjen City (TW)

Correspondence Address: NAN CHIH LU, 235 CHUNG-HO BOX 8-24, TAIPEI, TW

(21) Appl. No.: 09/885,765

(22) Filed: Jun. 19, 2001

(51) Int. Cl. A61F 7/00

(52) U.S. Cl. 607/114

(57) ABSTRACT

The present invention Water Bag with New Structure for Cold/Hot Hydrotherapy is designed to allow users to pour either hot water or cold water into the bag for the purpose of hydrotherapy. The water bag is made up of top layer, bottom layer, and cover. The top layer and the bottom layer are made in different thickness so as to provide different temperatures for users' choice. Additionally, the present invention incorporates the dual leaking-proof design to prevent the water from leaking. Therefore, the present invention is entirely different from the conventional water bags, and is an invention of breakthrough.

### BACKGROUND OF THE INVENTION

1. Field of the Invention

The present invention relates to a water bag with new structure for cold/hot hydrotherapy wherein the water bag provides two temperatures on both sides for the users' choice, and the dual leaking-proof design upgrades the safety of the water bag for users' protection.

In an effort to provide two temperatures on both sides, the inventor has designed both sides of the water bag in different thickness.

For the purpose of leaking-proof, a pressing-ring is disposed in the bottom of the cover, and a guiding-ring is disposed in the bottom of the ring. When the pressing-ring and the guiding-ring are tightened together, the water inside the bag will not be leaked at all. The water-resisting rim serves as an additional leaking-proof device in order to provide dual leaking-proof for the present invention.

2. Description of the Prior Art

The present invention relates to a water bag with new structure for cold/hot hydrotherapy referring to a brand new design of water bag, which provides two temperatures on both sides and dual leaking-proof design for users' utmost protection and convenience.

### SUMMARY OF THE INVENTION

The conventional water bags are made of rubber. The rubber layers on both sides are approximately 3˜7 mm thick. The temperature cannot circulate properly and, consequently, the water bag does not serve the purpose well. Secondly, users have hard time pouring water and ice into the water bag through the small water-inlet. Thirdly, the conventional water bag doesn't look well and can be leaking easily. These problems need to be solved for the users' convenience.

In an effort to solve the problems permanently, the inventor has studied the structure thoroughly, and believes that the present invention can serve the purposes well for users' convenience.

The value of present invention will be justified in regard to its technical content after reading the detailed description of the preferred embodiments of the present invention in reference to the accompanying drawings, wherein:

### BRIEF DESCRIPTION OF THE DRAWINGS

FIG. 1 is a three-dimensional drawing of present invention;

FIG. 2 is a three-dimensional sectional view showing a portion of present invention;

FIG. 3 is a cutaway view showing a portion of present invention;

FIG. 4 is another cutaway view showing a portion of present invention;

FIG. 5 is a reference view showing the use of present invention.

### DETAILED DESCRIPTION OF THE PREFERRED EMBODIMENT

Please refer to the preceding drawings together with the following description for the use of the present invention.

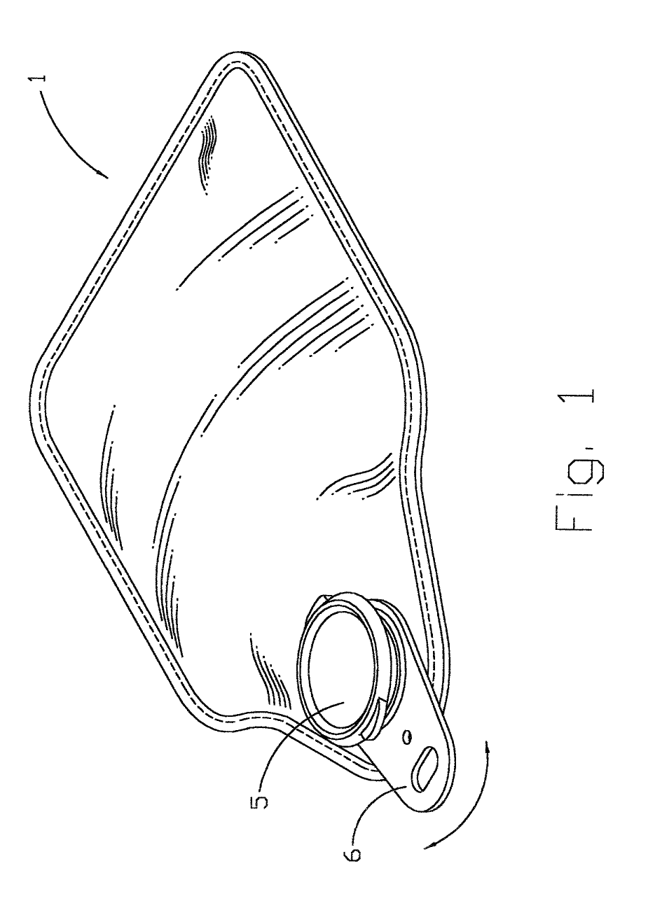

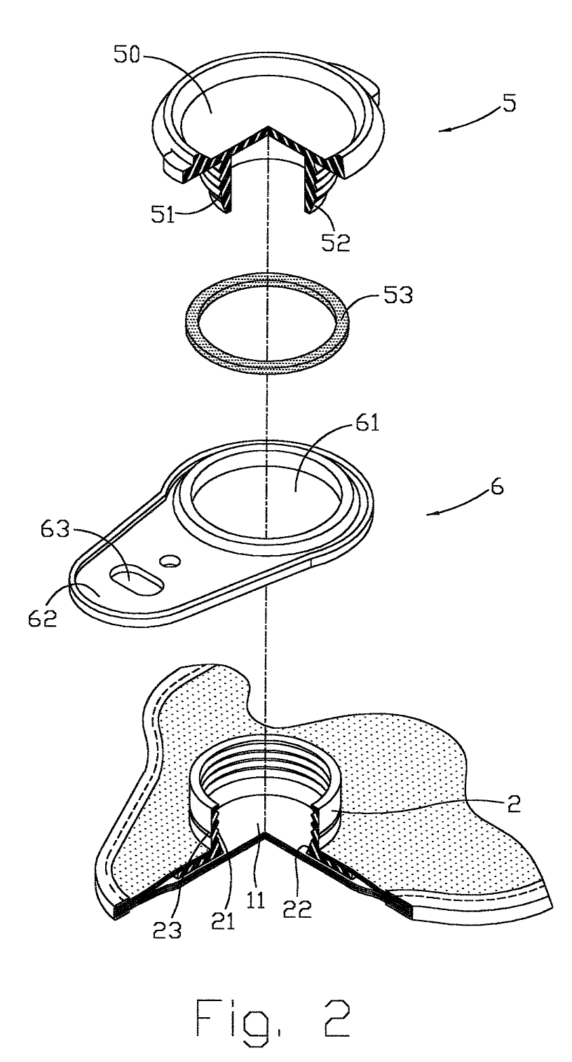

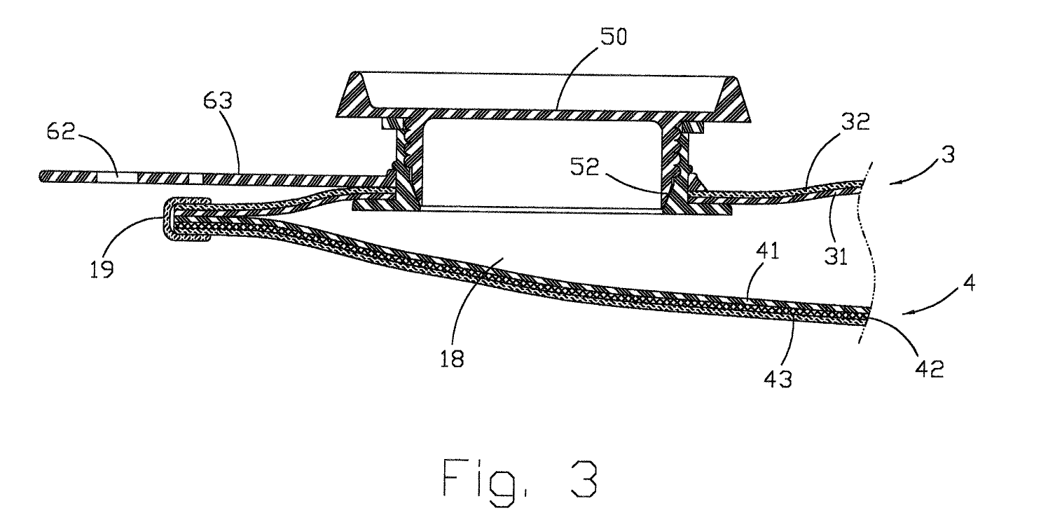

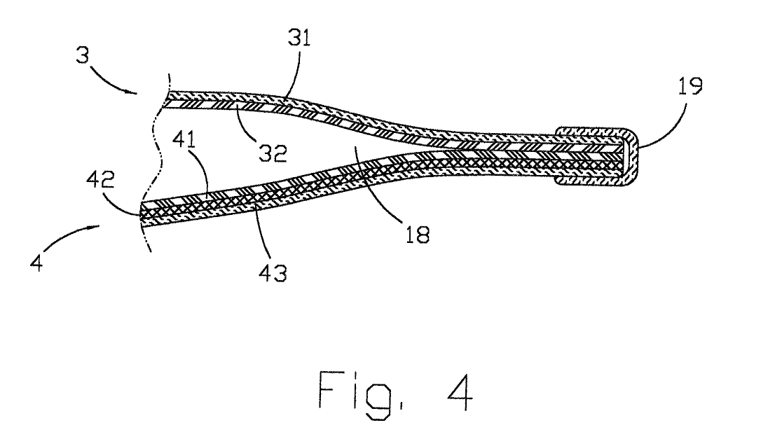

As shown by FIG. 1, FIG. 2, FIG. 3, and FIG. 4, the water bag 1 is made up of the ring 2, top layer 3, bottom layer 4, cover 5, and the connection object 6. The top layer 3 and the bottom layer 4 encircle the water-space 18, wherein:

The top layer 3 consists of water-resisting layer 31 and external cover 32. There is an opening 11 in the top layer 3, and a ring 2 next to the opening 11. Inside the ring 2 are spiral 21 and guiding-edge 22. Outside the ring 2 is a blocking-ring 23;

The bottom layer 4 consists of water-resisting layer 41 and external cover 43. There is a separation layer 42 between the water-resisting layer 41 and the external cover 43. The edges of the water-resisting layer 31 and the water-resisting layer 41 are pressed together with the high-wave technique and, therefore, the water-space 18 is created;

The cover 5 is disposed in the opening 11, and is made up of the top plate 50, external spiral 51, and pressing-ring 52;

The connection-object 6 connects to the ring 2 with the blocking-ring 23 stretching through the opening 61. There is an extending-plate 62 and a hole 63 in the connection-object 6. The extending-plate 62 serves as a handle for the users when they pour water into the water bag. The hole 63 allows the vendors to hang the water bag on the wall for sale.

In the present invention, both water-resisting layer 31 and the water-resisting layer 41 are made of PE. Manufacturers may use other materials as well. The manufacturers can use cotton, TC, or other materials to produce the external cover 32 and the external cover 43 so as to enhance the feeling of touch for users. The separation layer 42 can be made of nylon, cotton, cloth, or other materials.

As shown by the drawings, users may loosen the cover 5 of the ring 2; then pour cold water, hot water, or ice into the opening 11. As soon as the water-space 18 is full, users twist the cover 5 of the ring 2. The present invention incorporates dual leaking-proof design. This design consists of a water-resisting rim 53 and a unique sliding edge making use of the pressing-ring 52 and the guiding-edge 22 to keep water inside the bag. When users twist the cover 5 of the ring 2, the pressing-ring 52 and the guiding-edge 22 close tightly to prevent water from leaking.

The present invention is also designed to provide two temperatures on both sides of the water bag. The top layer 3 and the bottom layer 4 are different in thickness. When users pour hot water into the water bag 1, the top layer 3 generates higher temperature than the bottom layer 4 does. Users may choose either side of the water bag as they like. If the water is excessively hot, users may use the bottom layer 4 for some time; then switch to the top layer 3 as the water cool down. Apparently, the prevent invention serves the purpose better than the conventional ones.

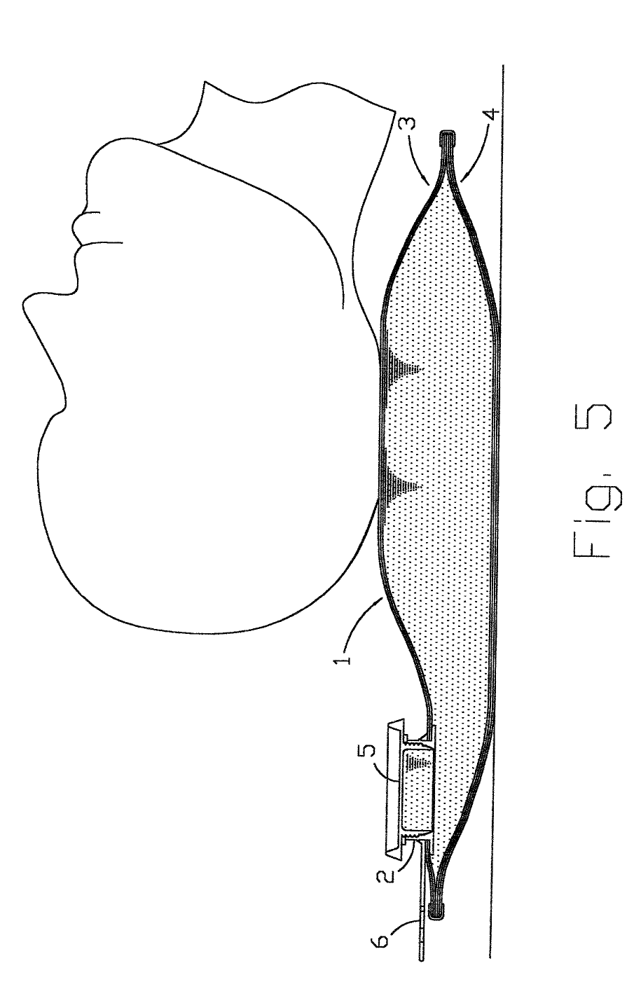

FIG. 5 is a reference drawing showing the use of the present invention.

As shown by the preceding description, the present invention is a valuable device for user's convenience, and has never been publicized in any document or market before. The inventor hereby presents his invention to the US Patents & Trademarks Office, and believes that his work meets the requirements of fashion, practicability, and progressiveness completely.

Having thus described my invention, what the inventor claims as new and desire to be secured by US Patents & Trademarks Office include:

1. A Water Bag with New Structure for Cold/Hot Hydrotherapy wherein the water bag consists of a ring, top layer, bottom layer, and cover, and there is a water-space between the top layer and the bottom layer. Of which:
a top layer comprising, to the minimum extent, water-resisting layer and external layer together with an opening in the top layer and a ring next to the opening;
a bottom layer comprising, to the minimum extent, water-resisting layer and external layer together with a separation layer between the water-resisting layer and the external layer; and
a cover disposed in the opening that allows users to open and close the water bag.

2. A Water Bag with New Structure for Cold/Hot Hydrotherapy as stated in claim 1, wherein the opening can be disposed in the bottom layer.

3. A Water Bag with New Structure for Cold/Hot Hydrotherapy as stated in claim 1, wherein the ring contains spirals and guiding-edge internally, and a blocking-ring externally.

4. A Water Bag with New Structure for Cold/Hot Hydrotherapy as stated in claim 1, wherein the cover is made up of top plate, spirals, and pressing ring.

5. A Water Bag with New Structure for Cold/Hot Hydrotherapy as stated in claim 1, wherein outside the ring is a connection-object that connects to the ring with the blocking-ring stretching through the opening.
